(19) United States
(12) Patent Application Publication
(10) Pub. No.: US 2009/0143785 A1
(43) Pub. Date: Jun. 4, 2009

(54) MEDICAL IMPACT TOOL ADAPTOR AND METHOD

(76) Inventors: Narissa Chang, Mansfield, MA (US); Linh Nguyen, Randolph, MA (US)

Correspondence Address: CARR LLP (IST), 670 FOUNDERS SQUARE, 900 JACKSON STREET, DALLAS, TX, 75202, US

(21) Appl. No.: 12/146,309

(22) Filed: Jun. 25, 2008

Related U.S. Application Data
(60) Provisional application No. 60/946,075, filed on Jun. 25, 2007.

(51) Int. Cl. A61B 17/88

(52) U.S. Cl. 606/99

(57) ABSTRACT

The present invention may provide for a medical impact tool adaptor for coupling with a medical impact tool and a method for applying an impact force to a medical instrument. The impact tool adaptor may comprise a main body and connector with a first attaching member and a second attaching member. The second attaching member may be pivotally coupled with the main body and may pivot between an opened position and a closed position relative to the first attaching member. The second attaching member may be releasably secured in a closed position via a movable lock mechanism. The first and second attaching members may be coupled to a medical tool via corresponding first and second attaching interfaces. In some embodiments, the impact tool adaptor may be pivotally coupled to the medical tool. Further, the impact tool adaptor may be releasably and/or pivotally coupled to the impact tool.

### CROSS-REFERENCED APPLICATIONS

This application relates to and claims priority from U.S. Provisional Patent Application Ser. No. 60/946,075 entitled “MEDICAL IMPACT TOOL ADAPTOR AND METHOD,” filed Jun. 25, 2007, and U.S. Utility patent application Ser. No. 12/018,913, entitled “ADAPTABLE TOOL REMOVAL DEVICE AND METHOD,” filed Jan. 24, 2008; and relates to U.S. patent application Ser. No. 11/690,692 entitled INSTRUMENTS FOR DELIVERING SPINAL IMPLANTS, filed Mar. 23, 2007, the entire contents of all of which applications are incorporated herein by reference for all purposes.

### BACKGROUND OF THE INVENTION

### Field of the Invention

The present invention relates to medical instruments and, more particularly, to medical impact tool instrument adaptors.

During the course of invasive medical and surgical procedures, medical tools and instruments may become trapped or caught by interior surfaces of a patient's body (e.g., between bony surfaces, among others). In addition, impact forces may be needed for insertion, removal, or repositioning of implants or medical devices, among other situations. In one illustrative situation, current methods of removing a seized medical instrument involve specifically designed removal tools configured to be attached to the particular seized instrument and other handheld devices such as hammers. Depending upon the procedure performed, an operating staff may have to maintain an inventory of medical instruments and a corresponding inventory of specific removal tools for each of the instruments. In addition, a surgeon may have to release the seized instrument in order to attach the removal tool or to apply an impact force via a separate hammer. An adaptable medical impact tool is needed that is readily attachable to a variety of seized medical instruments, and operable while retaining control of the seized instrument.

### SUMMARY

The present invention provides a medical impact tool adaptor that may comprise a first member, a second member, and a lock member. The second member may pivot between a first position and a second position with respect to the first member. The lock member may maintain the second member in one of the first position and the second position.

The present invention provides a method of applying an impact force to a medical tool by coupling a impact tool adaptor to a impact tool. The next step is to actuate a lock member to release a second member from a closed position. Further, the medical tool is engaged by coupling the first and second member to the medical tool. Following this, the second member is locked in place relative to the first member. Additionally, a sliding hammer is impacted against a stop at an end of the impact tool in order to transfer an impact force to the medical tool via the coupling between the impact tool, the impact tool adaptor, and the medical tool.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 illustrates a top perspective view of a impact tool adaptor in accordance with an embodiment of the present invention;

FIG. 2A illustrates a bottom perspective view of a side of the main body of the impact tool adaptor of FIG. 1,

FIG. 2B illustrates a bottom perspective view of another side of the main body of the impact tool adaptor of FIG. 1,

FIG. 3A illustrates an exploded perspective assembly view of a first attaching member;

FIG. 3B illustrates a side cross-sectional view of the first attaching member of FIG. 3A;

FIG. 4A illustrates an exploded perspective assembly view of a second attaching member;

FIG. 4B illustrates a side cross-sectional view of the second attaching member of FIG. 4A;

FIG. 5 illustrates a perspective view of a lock;

FIG. 6 illustrates an exploded perspective assembly view of the impact tool adaptor of FIG. 1;

FIG. 7 illustrates a cross-sectional side view of a impact tool adaptor in an opened configuration;

FIG. 8 illustrates a cross-sectional side view of a impact tool adaptor in a closed configuration;

FIG. 9 illustrates a cross-sectional side view of a impact tool adaptor coupled to a impact tool; and

FIG. 10 illustrates a perspective view of a impact tool adaptor coupled to a impact tool and partially engaged to a medical tool.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail. Additionally, for the most part, minor details have been omitted inasmuch as such details are not considered necessary to obtain a complete understanding of the present invention, and are considered to be within the understanding of persons of ordinary skill in the relevant art.

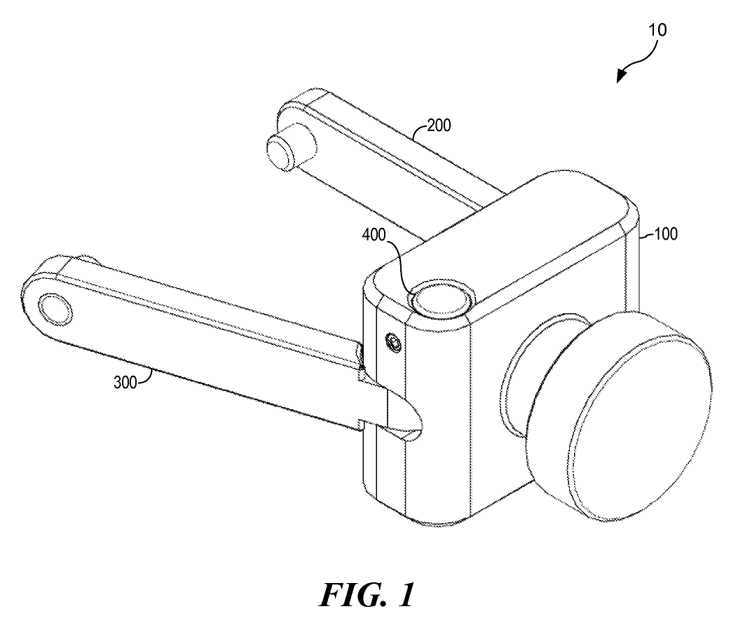

Turning now to FIG. 1, the reference numeral 10 generally indicates an illustrative embodiment of a impact tool adaptor 10 of the present invention. The impact tool 10 may comprise a main body 100, first attaching member 200, a second attaching member 300, and a lock 400. The first attaching member 200 may be secured to the main body 100 such that the first attaching member 200 is fixed or stationary with regard to position and orientation relative to the main body 100. In other embodiments, the first attaching member 200 may be integrally formed together with the main body 100. The second attaching member 300 may be pivotally coupled with the main body 100. The pivotal coupling of the second attaching member 300 may be configured such that the distal ends of the first and second attaching members 200, 300 may approach and withdraw from one another. The second attaching member 300 may be retained in a closed position due to the lock 400. The lock 400 may function to permit or restrain the motion of the second attaching member 300 depending upon the position of the lock 400 relative to the main body 100. The individual components of the impact tool adaptor 10 will be described in more detail in the following.

### Main Body

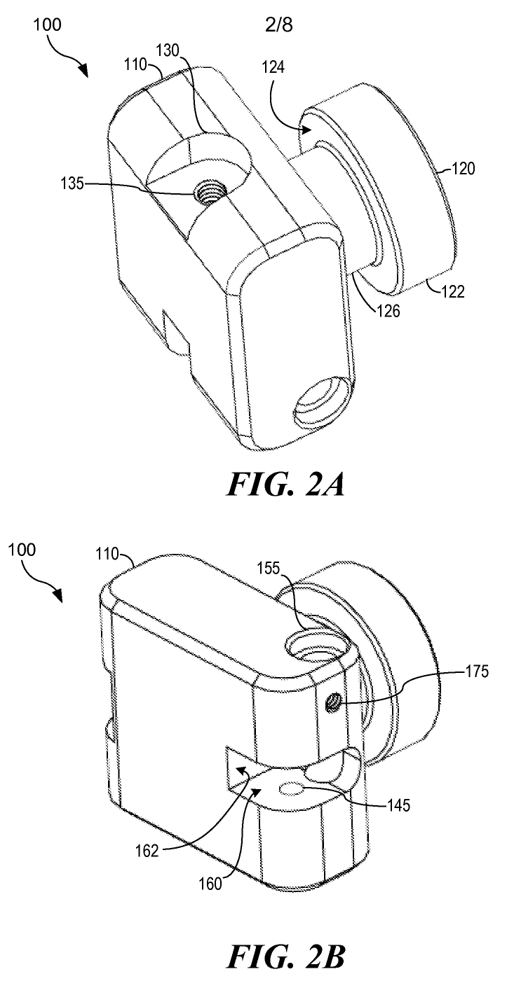

Referring now to FIG. 2A, the main body 100 may be formed from metal such as stainless steel, aluminum, and titanium, for example, from a variety of known processes, such as forging, sintering, machining, casting, or a combination of known processes. The main body 100 may comprise a lower body 110 and a connector 120. The lower body 110 may be integrally formed with the connector 120. In some embodiments, the connector 120 may be coupled to the lower body 110 through a mechanical fastener, chemical adhesive and/or bonding, welding, soldering, or any of a variety of known process for coupling together two metallic components.

The connector 120 may be substantially cylindrically shaped depending on the corresponding attachment configuration of the main impact tool, discussed later. The connector 120 may comprise a first cylinder 122 and a second cylinder 126. The first cylinder 120 may comprise a larger diameter than the second cylinder 126. The difference in relative diameters between the first and the second cylinders 122, 126 may form an indented neck or groove for attaching to the main impact tool. The cylindrical shape of the connector 120 may allow for rotation of the impact tool adaptor 10 relative to the main impact tool. However, cylinders are described for the connector 120 for the purposes of illustration only, the actual configuration may be any of a number of known geometric shapes or shapes otherwise conforming to the attachment configuration of the main impact tool, such as square, polygonal, or cylindrical with keyway or protruding member, for example. An advantage of using a non-cylindrical shape would be to inhibit rotation between the impact tool adaptor 10 and the main impact tool.

The use of a smaller diameter for the second cylinder 126 may form a connector abutment surface 124 between the first cylinder 122 and an opposing surface of the lower body 110. The abutment surface 124 may lie substantially within a single plane and may be configured to withstand repeated impact forces from the main impact tool. As shown in this illustrative embodiment, the abutment surface 124 comprises an area approximately equal to an area defined by the diameter of the first cylinder 122 minus an area defined by the diameter of the second cylinder 126.

The lower body 110 may further comprise a first recess 130 configured to correspond to a proximal end of the first attaching member 200 (FIG. 1). The first recess 130 may extend to a depth approximately equal to the thickness of the first attaching member 200 so that the first attaching member 200 may be coupled to the lower body 110 in a substantially flush state (i.e., in which an outer surface of the first attaching member 200 is substantially within a plane defined by the surface of the lower body 110 surrounding the first recess 130). The first recess may further comprise a first recess orifice 135 for threadably securing the first attaching member 200 to the lower body 110. As stated before, in some embodiments the first attaching member 200 may be integrally formed with the lower body 110 and not require any separate recess or attachment methods.

Turning now to FIG. 2B, the lower body 110 of the main body 100 may comprise a second recess 160 configured to pivotally accommodate a proximal end of the second attaching member 300 (FIG. 1). The shape of the second recess 160 may comprise one or more second recess walls 162 configured to prevent or inhibit the rotation of the second attaching member 300 beyond an intended angular range. In addition, the second recess 160 may be in communication with a pivot orifice 145 and a lock orifice 155. The pivot orifice 145 may be configured to accommodate a pivot pin, explained later. The pivot pin may be coupled to the pivot orifice 145 via pressing, welding, soldering or retained through the use of mechanical fasteners or retention clips for example. The pivot pin may define a rotational axis for the second attaching member 300. The lock orifice 155 may be configured to accommodate the lock 400 (FIG. 1), explained later. The lock 400 may function within the lock orifice 155 to permit or inhibit the pivoting of the second attaching member 300. In this illustrative example, the lock orifice 155 may substantially comprise two concentric cylinders in which the cylinder proximate to a surface of the lower body 110 may be larger than the cylinder further within the lower body 110. One or both of the concentric cylinders of the lock orifice 155 may extend into and interfere with at least a portion of the second recess 160. A retention orifice 175 may extend into and interfere with at least a portion of the lock orifice 155. In this illustrative example, the retention orifice 175 may threadably accommodate a retention member explained later.

### First Attaching Member

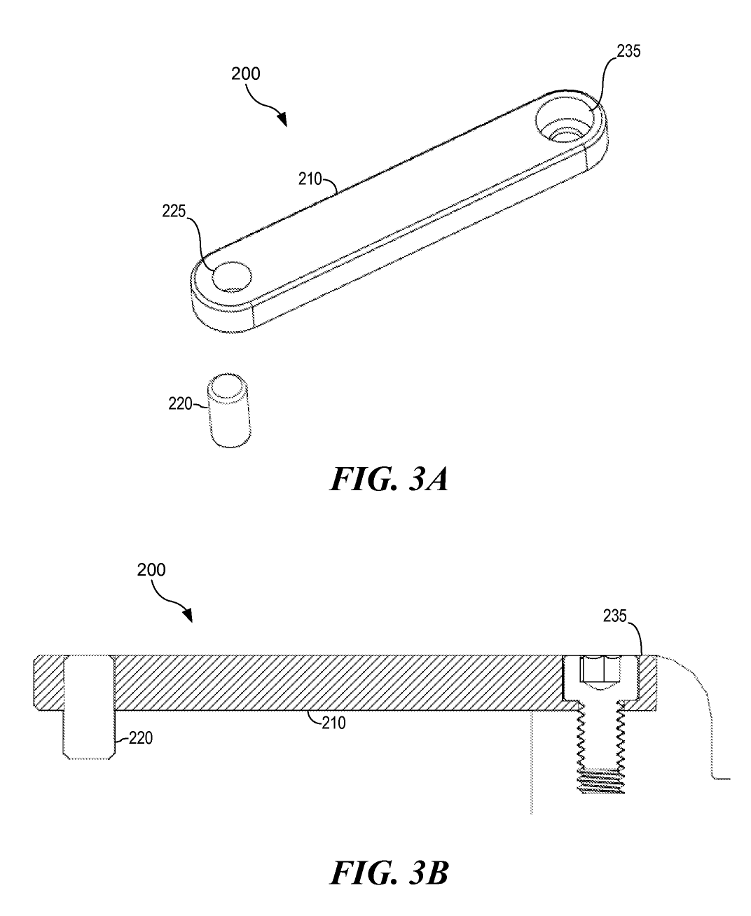

Referring now to FIGS. 3A and 3B, the first attaching member 200 may comprise a first attaching body 210 and a first attaching interface 220. The first attaching body 210 may comprise a proximal end containing a first attaching fastening orifice 235 and a distal end containing a first attaching interface orifice 225. The first attaching interface orifice 225 may accommodate the first attaching interface 220. The first attaching interface 220 may be substantially cylindrical for example, but is not required to be so configured. An advantage of using a substantially cylindrical shape for the first attaching interface 220 may be to allow for the rotation of the impact tool adaptor 10 relative to a medical tool requiring an impact force. The first attaching interface 220 may be configured to couple with correspondingly shaped components secured to the medical tool requiring an impact for adjustment, insertion, or removal, for example. The first attaching interface 220 may be secured to the first attaching body 210 through a press fit, chemical or mechanical fastening, welding, soldering, or any of a variety of known attachment methods. In this illustrative embodiment, the first attaching member 200 may be shown as a substantially straight, plate shaped, elongated member. However, the first attaching member 200 may not be limited to this configuration. The first attaching member 200 may be arcuate, cylindrical, or any of a variety of shapes, configurations, and orientations able to connect to the medical tool requiring an impact for adjustment, insertion, or removal, for example.

### Second Attaching Member

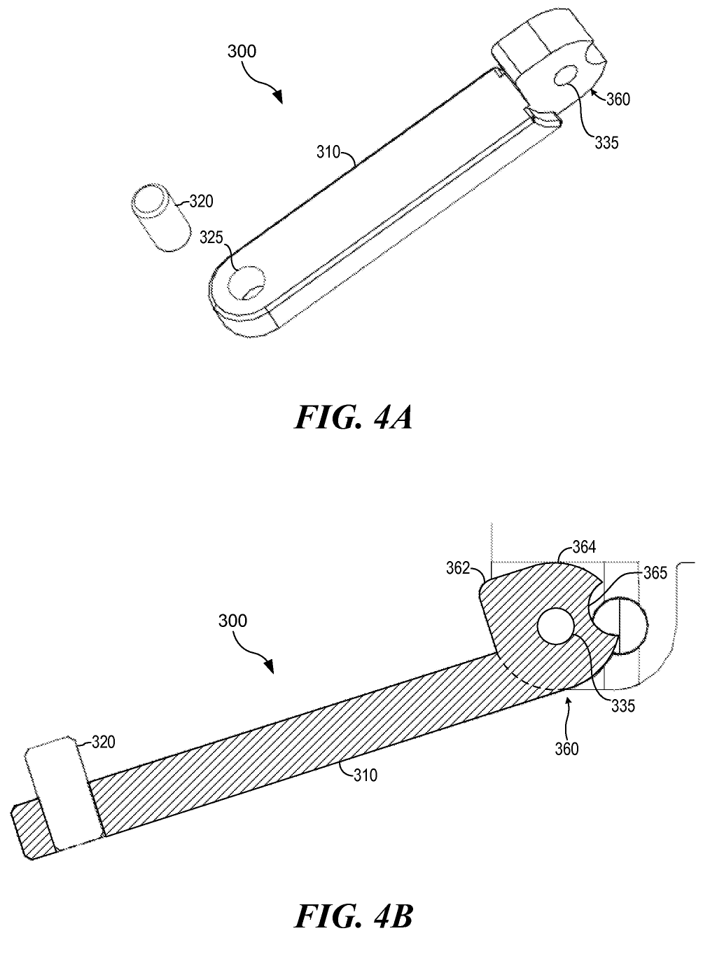

Turning now to FIGS. 4A and 4B, the second attaching member 300 may comprise a second attaching body 310 and a second attaching interface 320. The second attaching body 310 may comprise a proximal end 360 containing a second attaching fastening orifice 335 and a distal end containing a second attaching interface orifice 325. The second attaching interface orifice 325 may accommodate the second attaching interface 320. The second attaching interface 320 may be substantially cylindrical for example, but is not required to be so configured. An advantage of using a substantially cylindrical shape for the second attaching interface 320 may be to allow for the rotation of the impact tool adaptor 10 relative to a medical tool requiring an impact force. The second attaching interface 320 may be configured to couple with correspondingly shaped components secured to the medical tool requiring an applied impact force. The second attaching interface 320 may be secured to the second attaching body 310 through a press fit, chemical or mechanical fastening, welding, soldering, or any of a variety of known attachment methods. In this illustrative embodiment, the second attaching member 300 may be shown as a substantially straight, plate shaped, elongated member. However, the second attaching member 300 may not be limited to this configuration. The second attaching member 300 may be arcuate, cylindrical, or any of a variety of shapes, configurations, and orientations able to connect to the medical tool requiring an impact force.

The proximal end 360 of the second attaching body 310 may be transversely oriented (i.e., substantially orthogonal) to the distal end of the second attaching body 310. The proximal end 360 may comprise a first abutment surface 362 and a second abutment surface 364. The first abutment surface 362 may be substantially planar while the second abutment surface 364 may be substantially arcuate. The first and the second abutment surfaces 362, 364 may interact with the interior walls of the second recess 160 (FIG. 2B) to define a pivotal range of motion for the second attaching member 300. The second attaching member 300 may pivot about the second attaching fastening orifice 335. The second abutment surface 364 may be centered about a central axis of the second attaching fastening orifice 335. The proximal end 360 of the second attaching body 310 may further comprise an arcuate bolt recess 365 located in the second abutment surface 364. The arcuate bolt recess 365 may substantially correspond to an outer diameter of a concentric cylinder of the lock 400 (FIG. 1), explained later. The interaction between the arcuate bolt recess 365 and the lock 400 may inhibit or restrain the second attaching member 300 in a closed position, substantially parallel to the first attaching member 200 (FIG. 1). The thickness of the proximal end 360 of the second attaching portion 300 may slidably interact with one or more interior surfaces of the second recess 160.

### Lock

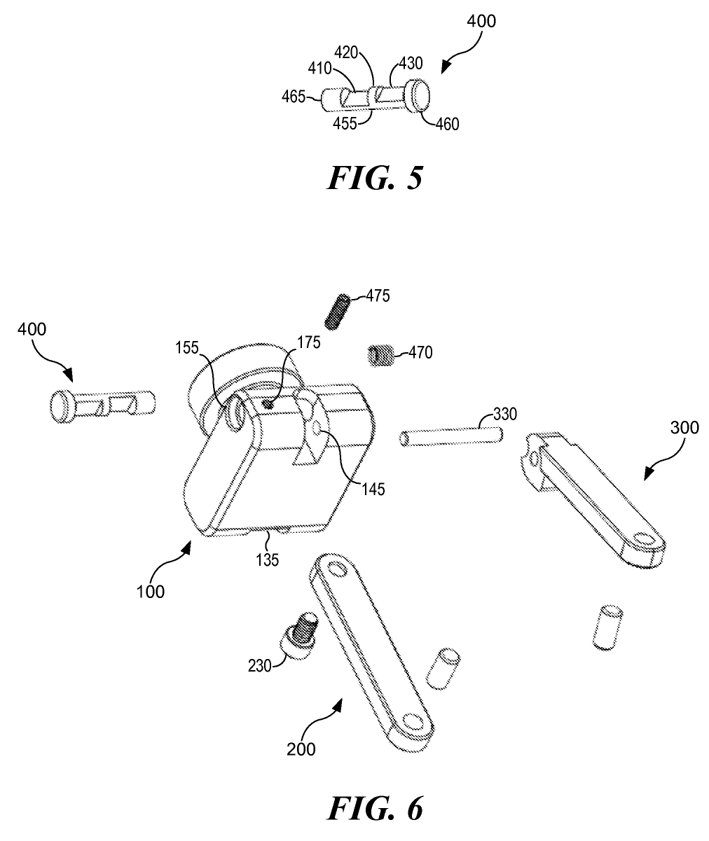

Turning now to FIG. 5, lock 400 comprises a locking portion 455 and an actuation portion 460. The locking portion 455 and the actuation portion 460 may substantially be in the form of concentric cylinders placed end to end. The actuation portion 460 may be larger in diameter than the locking portion 455 and may be configured to be pressed in order to inhibit and release the movement of the second attaching member 300. The outer diameters of the locking portion 455 and the actuation portion 460 may be configured to slidably fit within the lock orifice 155. Although concentric cylinders are shown in this illustrative example, the lock 400 is not to be limited to this exemplary embodiment. The lock 400 may be formed in a variety of shapes and configurations capable of interacting with the second attaching member 300 in the appropriate manner.

The locking portion 455 may further comprise a pivotal recess 410 and a retention recess 430. The pivotal recess 410 may be separated from the retention recess 430 by a cylindrical wall 420. In addition, a bolt portion 465 may be located at the distal end of the locking portion 455. The pivotal recess 410 and the retention recess 430 may be in the form of a slot or keyway cut into the locking portion 455 of the lock 400. The pivotal recess 410 and the retention recess 430 may be shown on the same side of the lock 400 but the lock 400 may not be limited to this configuration. The pivotal recess 410 may be shallower or deeper than the retention recess 430 and/or may be on the same side of the lock 400 or at an angle to one another. The bolt portion 465 may be a substantially unmodified section of the locking portion 455. In such a case, the bolt portion 465 may be configured essentially as a solid cylindrical section of the locking portion 455. Although the bolt portion 465 is shown as having the same general configuration as the rest of the locking portion 455, the bolt portion 465 may be larger or smaller in circumference, or machined into another configuration. The bolt portion 465 may correspond to the bolt recess 365 located in the second attaching member 300 (FIG. 4B).

The pivotal recess 410 may be positioned next to the bolt portion 465 for a linearly actuated lock 400. In some embodiments comprising a rotatably actuated lock (not shown), the pivotal recess 410 may be positioned on the other side of the lock 400 opposite to a central axis. The pivotal recess 410 provides clearance for the second attaching member 300 to pivot relative to the main body 100 (FIG. 1), this is explained later in more detail. The bolt portion 465 interacts with the bolt recess 365 located in the proximal end 360 of the second attaching member 300 (FIG. 4B) to rotatably fix the second attaching member 300 in a closed position.

### Assembly

Turning now to FIG. 6, the first attaching member 200 may be mechanically fastened to the main body 100 via a first threaded fastener 230. As stated before, the first attaching member 200 may be coupled to the main body 100 through a variety of methods known to people of skill in the art. These methods include, but are not limited to, welding, soldering, mechanically fastening, chemically bonding or adhering, or integrally forming or machining the attaching member 200 from a solid piece of material that includes the main body 100. The attaching member 200 may be coupled to the main body 100 so as to be fixed in position and orientation relative to the main body 100.

The second attaching member 300 may be pivotally secured to the main body 100 via the pivot pin 330. The pivot pin 330 may be secured to the main body through the use of an interference fit or any of a variety of known attachment methods, including but not limited to mechanical fastening, chemically bonding or adhering, welding or soldering, among others. Some embodiments may also comprise a bearing member intermediate to the pivot pin 330 and the second attaching member 300, or intermediate to the pivot pin 330 and the main body 100. The pivot pin 330 may be inserted into the pivot orifice 145 located in the main body 100. Further, the pivot pin 330 may be inserted into the second attaching fastening orifice 335 of the proximal end 360 of the second attaching member 300. In some embodiments, a resilient member (not shown) may be interfaced between the second attaching member 300 and the main body 100 so as to bias the second attaching member 300 in an outward direction (e.g., away from the first attaching member 200). In other embodiments, the resilient member may be interfaced between the second attaching member 300 and the main body 100 so as to bias the second attaching member 300 in an inward direction (e.g., toward the first attaching member 200). For example, the resilient member may be a torsion spring located about the pivot pin 330 and coupled to the second attaching member 300 and the main body 100.

The lock 400 may be inserted into the lock orifice 155 of the main body 100. Prior to insertion of the lock 400, a resilient member 470 may be inserted into the base of the lock orifice 155. The resilient member 470, shown in the form of a coil spring but not limited to this configuration, provides an outward bias against the lock 400. The lock 400 may be retained within the lock orifice 155 by the lock retainer 475. The lock retainer 475 may be threadably inserted into the retention orifice 175 so as to engage the retention recess 430 of the lock 400. In this illustrative embodiment, the lock retainer 475 engaging the retention recess 430 of the lock 400 may permit translation of the lock 400 through a range of motion, but inhibit the rotation of the lock 400 relative to the main body 100. The lock retainer 475 abutting either end of the retention recess 430 may define the range of motion of the lock 400. At one end of the range of motion of the lock 400, the bolt portion 465 of the lock may be aligned with and engage the bolt recess 365. At the other end of the range of motion of the lock 400, the second abutment surface 364 may be aligned with and engage the pivotal recess 410 of the lock 400.

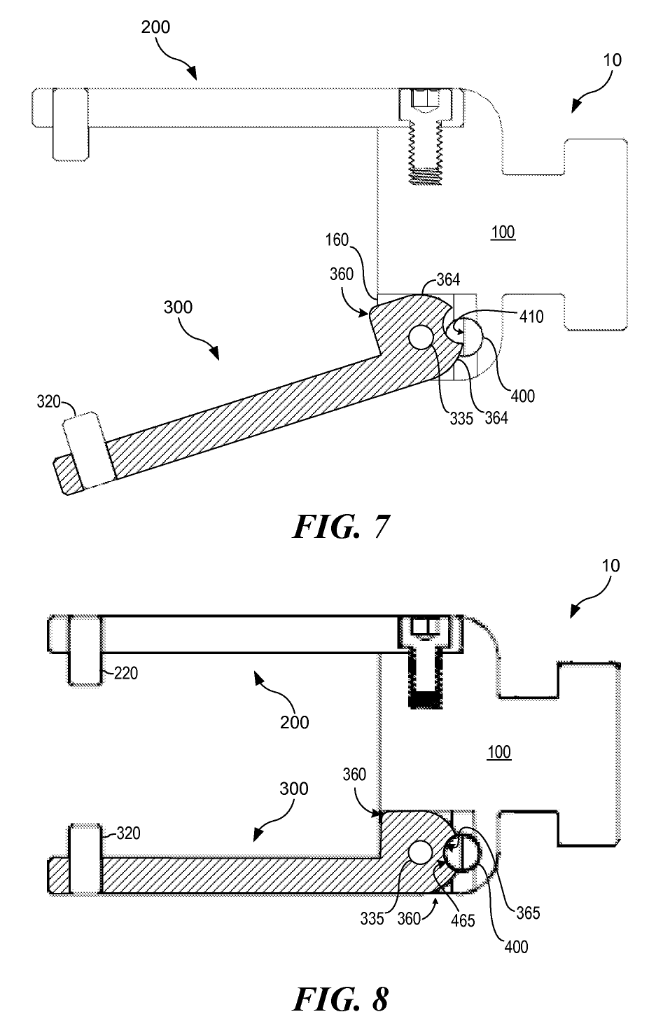

Turning now to FIG. 7, this cross-sectional view illustrates an interaction between the lock 400 and the second attaching member 300 when the second attaching member 300 is in an opened configuration. The lock 400 may be pressed inward (into the plane of the drawing) against the bias of the resilient member 470 (FIG. 6), relative to the main body 100, substantially aligning the second abutment surface 364 with the pivotal recess 410. The lock 400 may be held in place due to an interaction between the side of the proximal end 360 of the second attaching member 300 abutting the opposing side of the slot forming the pivotal recess 410. Accordingly, the lock 400 may be held in an inserted position against the bias of the resilient member 470.

The second attaching member 300 may be pivoted in an open configuration in which the second attaching interface 320 is withdrawn from the first attaching interface 220 of the first attaching member 200. The range of pivoting of the second attaching member 300 may be defined by an interaction between the second abutment surface 364 and one or both of an inner wall of the second recess 160 or a surface defining the pivotal recess 410. The second attaching member 300 may pivot about a central axis defining the second attaching fastening orifice 335 located in the proximal end 360 of the second attaching member 300.

Referring now to FIG. 8, this cross-sectional view illustrates an interaction between the lock 400 and the second attaching member 300 when the second attaching member 300 is in a closed configuration. The lock 400 may be extended to an outward limit defined by the range of motion permitted due to the engagement between the retention recess 430 (FIG. 5) and the lock retainer 475 (FIG. 6). The lock 400 may be extended outward and retained in this position as a result of the bias provided by the resilient member 470 (FIG. 6).

The movement of the lock 400 may result in the bolt portion 465 sliding into alignment and engagement with the lock recess 365 of the proximal end 360 of the second attaching member 300. The engagement between the bolt portion 465 and the lock recess 365 may inhibit or prevent the second attaching member 300 from moving between a closed and an opened position. The second attaching member 300 may be releasably locked in a closed position (i.e., a position in which the second attaching member 300 may be substantially parallel to the first attaching member 200 and the second attaching interface 320 may be proximate to the first attaching interface 220. The motion of the second attaching member 300 in moving to a closed position may be limited by the interaction between the first abutment surface 362 and an inner wall of the second recess 160.

### Use of the Impact Tool Adaptor

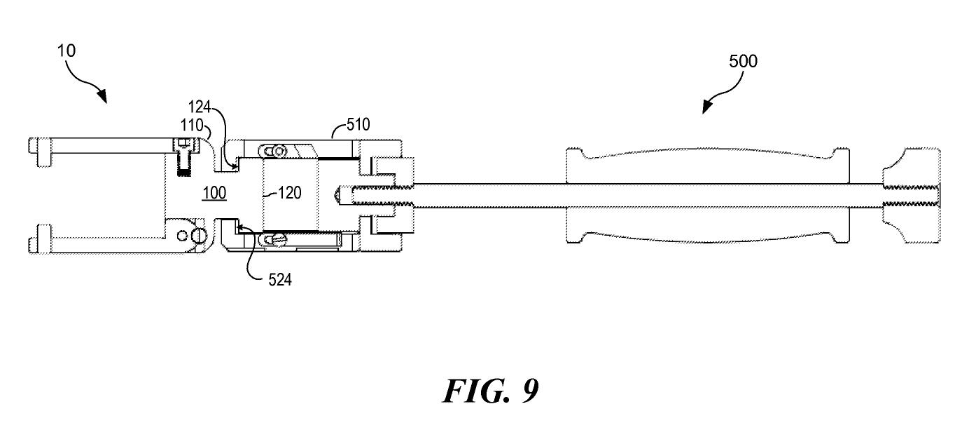

Turning now to FIG. 9, this figure illustrates a cross-section of a impact tool adaptor 10 coupled with a impact tool 500, such as the impact tool 500 described in co-pending U.S. Provisional Patent Application Ser. No. 60/886,589 entitled “ADAPTABLE TOOL REMOVAL DEVICE AND METHOD,” the contents of which are incorporated herein. The impact tool adaptor 10 may be pivotally coupled to a impact tool 500 about the connector portion 120 of the main body 100.

The distal end of the impact tool 500 may comprise first and second connector members 510 (only one set of connecting members may be seen in this figure). The first and second connecting members 510 may move between an open and closed position. In the closed position, the first and second connecting members 510 may form a tool abutment surface 524 corresponding to and opposing the abutment surface 124 of the impact tool adaptor 124. The distal ends of the first and second connecting members 510 may be constrained between the first cylinder 122 of the connector 120 and the lower body 110 of the main body 100.

In some embodiments in which the distal ends of the first and second connecting members 510 are positioned about a second cylinder 126, the impact tool adaptor 10 may be able to rotate relative to the impact tool 500. In other embodiments, the impact tool adaptor 10 may be constrained to maintain a fixed orientation with regard to the impact tool 500.

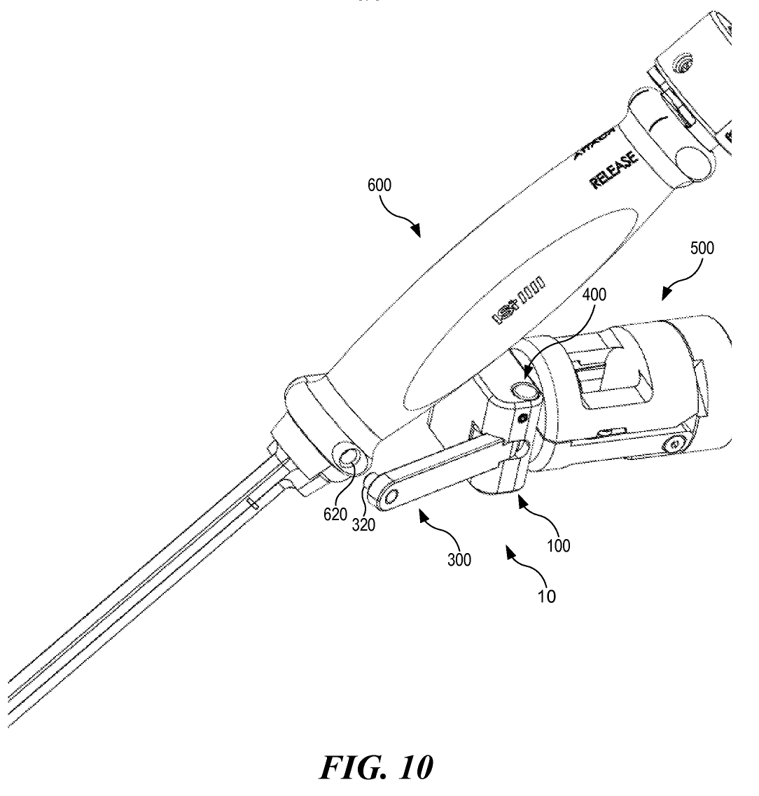

Referring to FIG. 10, the impact tool adaptor 10, attached to a impact tool 500, may be coupled to a medical tool 600 through the first and second attaching interfaces 220, 320 of the first and second attaching members 200, 300. The lock 400, may be position in an outward position, aligning the pivotal recess 410 with the proximal end 360 of the second attaching member 300 (see FIG. 7). The second attaching member 300 may then move to an open position as shown in the figure.

The first attaching interface 220 of the first attaching member 200 may be inserted into a corresponding tool attaching interface (e.g., similar to the tool attaching interface 620 but not visible in this figure). In the illustrative embodiment shown, the first and second attaching interfaces 220, 320 may be configured substantially as cylinders and the corresponding tool attaching interfaces 620 may be configured substantially as cylindrical orifices. Of course, this is for the purposes of illustration only. In certain embodiments, the first and second attaching interfaces 220, 320 may comprise orifices located in the first and second attaching members 200, 300 and the medical tool 600 may comprise protrusions (instead of the attaching interface 620) configured to engage the orifices of the first and second attaching members 200, 300. The interaction between the first and second attaching interfaces 220, 320 and the corresponding tool attaching interfaces 620 may allow the assembly of the impact tool adaptor 10 and the impact tool 500 to rotate relative to the medical tool 600 about the central axes defining the first and second attaching interfaces 220, 320. However, the ability to rotate may be an advantage, but may not be a requirement of the impact tool adaptor 10. The first and second attaching interfaces 220, 320 and the corresponding tool attaching interfaces 620 may be configured in a variety of shapes and sizes, including but not limited to square, rectangular, conical, and spherical configurations for example. In such a case, the assembly of the impact tool adaptor 10 and the impact tool 500 may be substantially fixed in orientation relative to the medical tool 600.

After the first attaching interface 220 is coupled to the corresponding tool attaching interface (not visible in this figure), the operator may close the second attaching member 300. Closing the second attaching member 300 may couple the second attaching interface 320 and the corresponding tool attaching interface 620. As the second attaching interface 320 becomes fully engaged with the corresponding tool attaching interface 620, the bolt portion 465 of the lock 400 may be biased to automatically engage the lock recess 365. The second attaching member 300 may then be locked in a closed position relative to the first attaching member 200.

After locking the second attaching member 300 in a closed position, the impact tool 500 may be operated to apply an impact force to the medical tool 600. In some embodiments, operating the impact tool 500 may involve sliding a weighted hammer along a shaft until an impact with a stop located at an end of the shaft. At this point, the hammer may transfer an impact force from the stop, through the shaft, and into the impact tool adaptor 10 via an abutting interface between the abutment surface 124 and the tool abutment surface 524 (FIG. 9). The impact force may then be transferred via the interface between the first and second attaching interfaces 220, 320 and the tool connecting interfaces 620. Ultimately, the impact force may be transferred along the medical tool 600 to the distal end of the medical tool 600, such as a LIF (lumbar interbody fusion device) or rasp, among others.

Once the medical tool 600 is freed or released from being seized for example (e.g., among other situations requiring an applied impact force), the impact tool adaptor 10 may be uncoupled from the medical tool 600. In order to uncouple the impact tool adaptor 10, the lock 400 may be pressed inward relative to the main body 100, releasing the bolt portion 465 of the lock 400 from engagement with the lock recess 365. As the pivotal recess 410 becomes aligned with the proximal end 360 of the second attaching member 300, the second attaching member 300 becomes free to move from a closed position to an opened position. In some embodiments, a resilient member may automatically rotate the second attaching member 300 to an open position when the proximal end 360 is aligned with the pivotal recess 410. In the opened position, the second attaching interface 320 may be disengaged from the corresponding tool attaching interface 620. The first attaching interface 220 (FIG. 9) may then be disengaged form the corresponding tool attaching interface (not visible in this figure). The medical tool 600 may then be released from the impact tool adaptor 10.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

1. A tool adapter for cooperative operation with an impact tool for removing a medical instrument from a patient, comprising:
an attaching interface member fittable with a corresponding attaching interface member of an impact tool for applying an impact force to the tool adapter, and
first and second rotatable attaching interfaces fittable into corresponding attaching interfaces of the medical instrument such that attachment of the tool adaptor onto the medical instrument permits rotation of an impact tool attached to the tool adapter to a desired orientation about an axis extending through the first and second attaching interfaces of the tool adapter relative to the main axis of the medical tool aligned toward the patient, such that an impact force can be applied to the medical instrument other than along the main axis of the medical instrument.

2. The tool adapter of claim 1 wherein the attaching interface member of the tool adapter further comprises:
a head defining a head circumference larger than an accommodating circumference in the corresponding attaching interface member of the impact tool and
a neck defining a neck circumference smaller than an accommodating circumference in the corresponding attaching interface member of the impact tool.

3. A method for removing a medical instrument from a patient, the method comprising:
coupling a tool adaptor having first and second rotatable attaching interfaces to corresponding attaching interfaces of a medical instrument having its main axis aligned toward a patient;
coupling the tool adapter to a first clamping member of an impact tool;
coupling the tool adapter to a second clamping member of the impact tool;
securing the clamping members in place;
rotating an impact tool attached to the tool adapter about an axis extending through the first and second attaching interfaces of the tool adapter relative to the main axis of the medical tool aligned toward the patient to a desired orientation; and
receiving an impact force on an impact surface such that a portion of the impact force is transferred to the medical instrument.

4. The method of claim 3 wherein locking the clamping members in a closed configuration comprises: rotating a locking member.

5. The method of claim 3 further comprising:
opening the clamping members; and
decoupling the first clamping member from the tool adapter.

6. The method of claim 3 wherein opening the clamping members comprises:
pivoting a pivotal member.

7. The method of claim 3 wherein the receiving of an impact force on an impact surface further comprises positioning a weight such that the weight can impact the impact surface.

8. The method of claim 3, wherein the impact force is transferred to the medical instrument other than along the main axis of the medical tool aligned toward the patient.
